(19) United States
(12) Patent Application Publication
(10) Pub. No.: US 2026/0209162 A1
(43) Pub. Date: Jul. 23, 2026

(54) CONTINUOUS PROCESS FOR CRYSTALLIZING BIS (2-HYDROXYETHYL) TEREPHTHALATE AND CRYSTALLIZER USED THEREIN

(71) Applicant: CHEMPET SRL, Cerano (NO) (IT)

(72) Inventors: Guido FRAGIACOMO, Novara (IT); Roberto PROSERPIO, Mariano Comense (CO) (IT); Marco CASAROTTI, Galliate (NO) (IT); Gabriele NIPOTI, Tromello (PV) (IT)

(73) Assignee: CHEMPET SRL, Cerano (NO) (IT)

(21) Appl. No.: 19/143,537

(22) PCT Filed: Dec. 21, 2023
(86) PCT No.: PCT/IB2023/063109

§ 371 Date: Jun. 26, 2025

(30) Foreign Application Priority Data
Dec. 27, 2022 (IT) 102022000026853

(51) Int. Cl. C07C 67/52; C07C 67/56

(52) U.S. Cl. CPC C07C 67/52; C07C 67/56

(57) ABSTRACT

Crystallization process of BHET in which a BHET solution is introduced into a crystallizer equipped with an impeller positioned in the lower part of the crystallizer and coaxial to a draft tube, which in turn is coaxial to the internal wall of the tank, which defines an interspace within which a plurality of heat exchange plates are placed in which a cooling fluid circulates. Thanks to the thrust of the impeller, the solution flows inside the interspace where the solution is cooled with high efficiency, thanks to the high exchange surface with the heat exchange plates, without generating excessive turbulence of the liquid. A suspension rich in BHET crystals with a low content of fines is therefore formed, which is then discharged from the crystalliser and sent to the subsequent BHET processing steps.

The present invention concerns a continuous process for crystallizing bis(2-hydroxyethyl) terephthalate (BHET), and a crystallizer used therein.

Polyethylene terephthalate (PET) is a widely used semi-crystalline thermoplastic polyester with high strength and transparency, which has various applications thanks to its physical and chemical properties, particularly in packaging and fiber production. Global PET production reached a capacity of over 70 million tonnes (Mton) in 2017, of which approximately 15 Mton was produced in Europe, intended for manufacturing synthetic fibers (approximately 67%), bottles (approximately 23%) and packaging (10%).

PET does not pose any safety risk, but increase in consumption and accumulation in waste streams and its non-biodegradability generate environmental and economic concerns. Therefore, there is growing interest in PET recycling technologies.

PET is considered a polymeric material that can be easily recycled and its recycling is the most widespread among polymeric materials. The technologies can be grouped into two macro-categories: mechanical and chemical recycling.

Mechanical recycling mainly consists of obtaining PET flakes, through crushing and grinding previously selected waste, which are sent directly to extrusion to produce new articles. The main problems of this technology are due to heterogeneity of the solid waste and low quality of the final product, since PET worsens its mechanical properties with each recycling process.

Nonetheless, to date mechanical recycling is still the most used technology for the treatment of waste containing PET, although it does not allow obtaining a high quality material suitable for contact with food. A growing interest in chemical recycling technologies is noticed, as they comply with the principles of sustainable development, returning raw materials for the production of virgin PET, which is of course of much higher quality than mechanically recycled PET.

Chemical recycling involves decomposition of polyester using a reagent capable of depolymerizing the PET chains to obtain the starting monomers; the chemical depolymerization of PET is usually achieved by hydrolysis or methanolysis or glycolysis.

Hydrolysis depolymerizes PET into terephthalic acid (TPA) and monoethylene glycol (MEG) (also called ethylene glycol—EG) by reaction with water. Methanolysis degrades PET to dimethyl terephthalate (DMT) and MEG by reaction with methanol. Glycolysis causes depolymerization by reaction with MEG, to produce bis(2-hydroxyethyl) terephthalate (BHET) , an intermediate product that is formed in the first stage of PET production from the starting monomers (terephthalic acid and MEG). The BHET obtained from the depolymerization of recycled PET is often referred to as “r-BHET”.

At the end of the glycolysis reaction, BHET is obtained in the form of a solution in water or MEG or mixtures thereof. After filtration to separate suspended solid pollutants, BHET is crystallized by cooling, filtered and purified.

A particularly effective purification process, especially with respect to dyes present as contaminants in the raw product, is described in patent application WO 2021/124149, where the raw BHET, in the form of an aqueous solution, is subjected to oxidation and then to the action of an absorbent agent (for example an activated carbon or a silica) that is capable of removing oxidation products of dyes or other contaminants present in the raw BHET solution.

The Applicant has found that the PET depolymerization process with BHET production includes steps that are generally carried out continuously, with clear advantages from the point of view of productivity and plant management.

However, unlike the other steps of the overall process, the crystallization step of the BHET obtained from the depolymerization is generally carried out in batches, i.e. discontinuously, for example in a crystallizer where the BHET solution is introduced in a predefined quantity and then cooled to a temperature such as to cause precipitation of BHET in crystalline form (around 15° C.). Cooling is generally achieved via a jacket external to the crystalliser into which the cooling fluid is introduced. This process requires vigorous and continuous stirring of the BHET solution to promote heat exchange between the solution and the internal wall of the crystallizer. However, BHET in crystalline form tends to deposit in considerable quantities on the cold wall of the crystalliser, forming a layer of crystals (scaling) which hinders heat exchange.

Furthermore, the Applicant has found that excessively vigorous stirring of the suspension in which the BHET crystals accumulate causes fragmentation of the crystals and secondary nucleation that generates a high quantity of fine crystals. This makes the subsequent filtration of the suspension obtained at the end of crystallization difficult, because fine crystals decrease permeability of the solvent through the layer of crystals that deposit on the filter, with a consequent increase in filtration times.

On the other hand, an increase in the amount of fine crystals can be caused by a high cooling rate, which favours nucleation at the expense of crystal growth.

The Applicant has therefore faced the technical problem of providing a process for crystallizing BHET which can operate continuously and which allows BHET to be obtained in the form of crystals of adequate dimensions to guarantee a high purity of the product and to facilitate filtration of the same. For this purpose it is necessary to increase efficiency of the crystallization process, so as to avoid the use of excessive stirring speeds inside the crystallizer which cause high turbulence of the liquid. This reduces the risk of crystal breakage and formation of nucleation germs.

The applicant has found that this technical problem, and others which will be better illustrated below, can be solved by carrying out a BHET crystallization process in which a BHET solution is introduced into a crystalliser equipped with an impeller positioned in the lower part of the crystalliser and coaxial to a draft tube, which in turn is coaxial to the internal wall of the tank, which defines an interspace within which a plurality of heat exchange plates are placed in which a cooling fluid circulates. Thanks to the impeller thrust, the solution flows inside the interspace where it cools efficiently thanks to high exchange surface with the heat exchange plates, so as to form a suspension rich in BHET crystals, which is discharged from the crystallizer and sent to the subsequent BHET processing steps.

According to a first aspect, the present invention therefore concerns a process for crystallizing bis(2-hydroxyethyl) terephthalate (BHET) in a continuous manner, which comprises:

introducing a BHET solution into a crystallizer which comprises a tank, preferably substantially cylindrical in shape, equipped with an impeller positioned in a lower part of the tank and coaxial to a draft tube, arranged inside the tank, preferably coaxial to the internal wall of the tank, where the external wall of the draft tube defines, with the internal wall of the tank, an interspace within which a plurality of heat exchange plates are located;

rotating the impeller so as to generate a thrust of the solution from the bottom upwards, along the longitudinal development direction of the draft tube and consequently a flow of the solution from the top downwards inside the interspace, where the solution cools on contact with the plurality of heat exchange plates in which a cooling fluid circulates, at a temperature such as to cause crystallization of the BHET with formation of a BHET crystal suspension;

discharging the BHET crystal suspension from the crystallizer.

In a second aspect, the present invention concerns a crystallizer for crystallizing BHET from a solution thereof, which comprises:

a tank, preferably having a substantially cylindrical shape;

a draft tube, arranged inside said tank, an interspace between the internal wall of the tank and the external wall of the draft tube being defined within which a plurality of heat exchange plates is located;

an impeller positioned in the lower part of the tank, internally and coaxially to the draft tube;

a feeding duct for feeding the BHET solution into the tank;

a discharge duct for discharging the crystallized BHET in a suspension form from the tank.

A preferred embodiment of the aforementioned crystallizer is illustrated, by way of example and without limitation, with the aid of the attached figures in which:

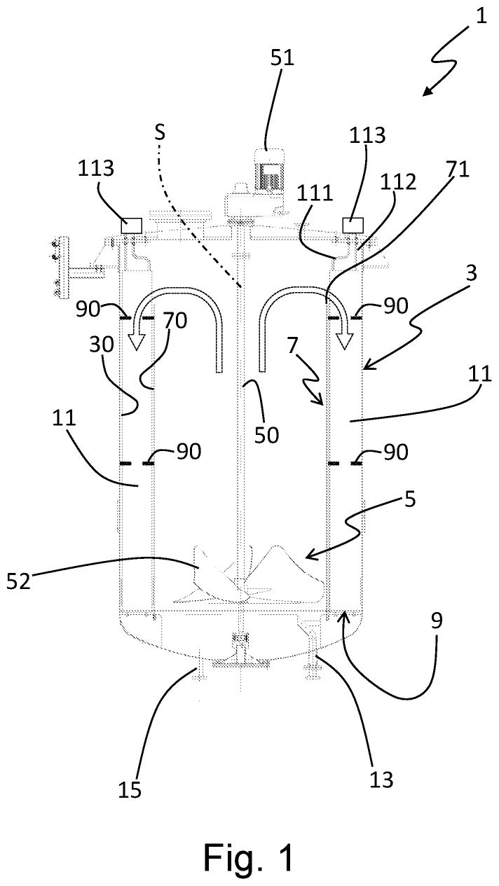

FIG. 1 is a sectional view along a vertical plane passing through the central axis of a crystallizer according to the invention;

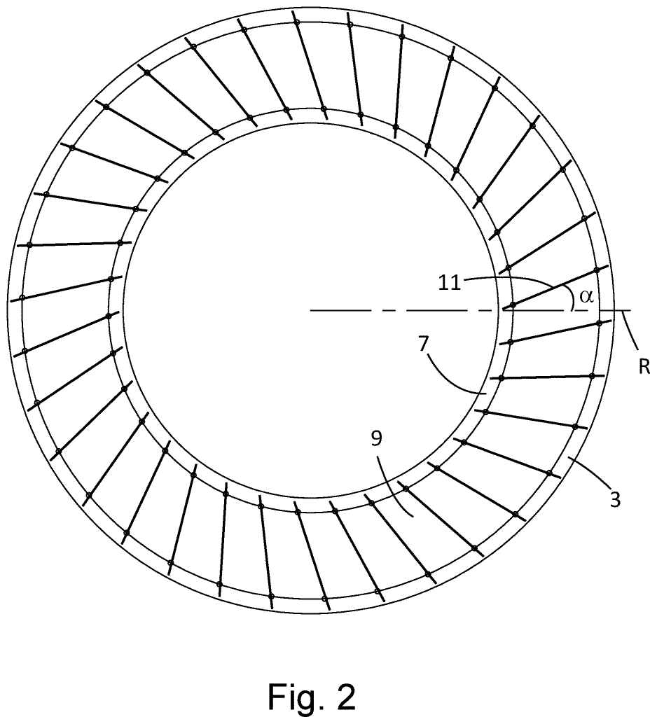

FIG. 2 is a sectional view along a transverse plane perpendicular to the central axis of the crystallizer of FIG. 1.

With reference to FIG. 1, the crystallizer (1) according to the present invention includes a tank (3), preferably having a substantially cylindrical shape, in which a draft tube (7) is inserted. In this way, between the internal wall (30) of the tank (3) and the external wall (70) of the draft tube (7) an interspace (9) is defined within which a plurality of heat exchange plates are placed (11). An impeller (5) is positioned in the lower part of the tank (3), internally and coaxially to the draft tube (7). There is also a feeding duct (13) for feeding the BHET solution into the tank (3), and a discharge duct (15) for discharging the crystallized BHET in a suspension form from the tank (3).

As regards the impeller (5), this preferably includes a rotation shaft (50), connected to a motor (51), at the end of which a plurality of blades (52) are fixed which are configured so as to generate, inside the draft tube (7), a thrust of the solution from the bottom upwards of the solution. In this way the solution is pushed upwards along the draft tube (7), up to the upper end (71) of the same, from where it flows down inside the interspace (9) with a flow from the top downwards.

In this regard, the Applicant has found that an impeller (5) which instead generates a thrust from top downwards (as happens for a common agitator) would not be suitable for the purposes of the invention, as it would cause formation of a vortex on the surface of the liquid, causing incorporation of air which disturbs the crystallization process, with formation of crystallization germs and therefore production of fine crystals.

Preferably, the impeller (5) is of the reverse helix type and is characterized by a high hydraulic flow rate and a low shear force, in order to delicately move even a significant volume of solution. The impeller (5) can optionally comprise a second plurality of blades (not shown in FIG. 1) connected to the rotation shaft (50) in an intermediate position between the first plurality of blades (52) and the upper end (71) of the draft tube (7). As regards the heat exchange plates (11), these can be placed inside the interspace (9) in a substantially radial direction.

Advantageously, the impeller (5) can be suitably sized and rotated at an angular speed such as to cause a solution flow having a relatively low speed, preferably between 0.2 m/sec and 2.0 m/sec, more preferably between 0.4 m/sec and 1.5 m/sec, inside the interspace (9) where the heat exchange plates (11) are placed.

Preferably each heat exchange plate (11) has a height, along the main direction of longitudinal development, at least equal to, preferably greater than, the height of the draft tube (7).

With reference to FIG. 2, i.e. a sectional view along a transverse plane perpendicular to the central axis of the crystalliser (1), preferably the heat exchange plates (11) are positioned inside the interspace (9) inclined, with respect to the corresponding radial direction (R), according to angles (α) between 10 and 50 degrees. The radial direction corresponding to a given plate is defined as the radial direction that intercepts the section of that plate, defined on the transverse plane, at its point closest to the central axis. In this way it is in fact possible to occupy the internal volume of the interspace (9) more effectively with a greater number of plates (11), so as to further increase efficiency of the crystallization process. In other words, the arrangement of the heat exchange plates (11) inclined with respect to the corresponding radial direction allows to increase the number of plates (11) that can be inserted inside the circular interspace (9), thus increasing the heat exchange surface available.

Again with reference to FIG. 1, the crystalliser (1) preferably comprises, inside the interspace (9), a plurality of guiding and holding elements (90) configured to keep the heat exchange plates (11) in position inside the interspace (9) itself.

According to a preferred embodiment, the heat exchange plates (11) have a pillow configuration, and are known in the industry as pillow plate heat exchangers. According to this configuration, each plate is made up of two metal plates welded together at the edges, which have an interspace between them having a wavy configuration. This interspace is formed by connection areas between the sheets (obtained for example by local welding) alternating with areas in which the two sheets swell, forming an interspace, into which the cooling fluid is introduced. This gives the plates the typical pillow configuration. Further details on the structure of such heat exchange plates are reported, for example, in patent applications DE 10 2016 005 999 A1 and CA 2 532 646A1 .

The heat exchange plates with pillow configuration are particularly advantageous for the present invention, as they have a complex wavy geometry that favours heat exchange with the fluid to be cooled, so as not to require vigorous stirring of the solution inside the crystallizer. In this way, the risk of breakage of the BHET crystals is minimized, at the same time reducing the formation of crystal deposits on the plates themselves (scaling).

In order to minimize the scaling phenomenon, the heat exchange plates preferably have the outer surface treated by electropolishing, which significantly reduces roughness of the metal surface and therefore formation of encrustations of crystalline material.

In the event that the heat exchange plates still have a significant deposit of crystals after a certain time of use, such as to affect heat exchange, it is possible to carry out a cleaning step of the plates, which includes interrupting the flow of cooling fluid and replace it with a flow of heating fluid. In this way the BHET crystals that deposited on the metal surface are redissolved, re-establishing an optimal heat exchange. The heating fluid, which can generally be water or a mixture of water and glycol, is preferably introduced into the heat exchange plates at a temperature of from 40° C. to 130° C., more preferably from 50° C. to 120° C.

In order not to interrupt continuity of the crystallization process, the cleaning step by heating can advantageously be carried out on a limited number of plates, preferably on a single plate, which are heated generating a local increase in temperature, which does not compromise crystallization on the other heat exchange plates that continue to be cooled.

Preferably, the crystalliser (1) comprises a number of heat exchange plates (11) greater than or equal to 20, more preferably greater than or equal to 30, even more preferably greater than or equal to 50. In this way efficiency of the crystallization process is maximized and a possible cleaning step of the plates is facilitated. In fact, cyclic heating of a single plate at a time, for cleaning it, does not have a significant influence on efficiency of the crystallization process.

Each heat exchange plate (11) includes an inlet conduit (111) and an outlet conduit (112) for the cooling fluid, or if necessary for the heating fluid.

Preferably, the crystallizer (1) comprises a plurality of solenoid valves (113), one for each heat exchange plate (11), configured to switch supply of each plate (11) between a cooling fluid supply and a heating fluid supply, for the cleaning step of the single plate (11) as described above.

Preferably the crystalliser (1) can comprise a heating jacket, placed externally to the walls of the tank (3) and suitable for heating the entire solution contained in the tank (3). In this way it is possible to homogeneously heat, if necessary, the entire internal volume of the tank (3), for example to carry out a quick cleaning which may become necessary following a plant stop.

As regards the temperature to which the solution is to be brought inside the crystalliser to obtain the BHET crystallization, this is generally between 10° C. and 40° C., preferably between 15° C. and 38° C.

Preferably, the crystallization process according to the present invention can be divided into at least two distinct crystallization steps.

A first step involves crystallizing the BHET from its crude solution, so as to obtain a suspension of BHET in crystalline form which is then filtered to obtain the BHET in crystalline form and a liquid phase (mother liquor).

A second step comprises: (i) redissolving the BHET in crystalline form obtained from the first step; (ii) subjecting the BHET solution thus obtained to a purification step, so as to obtain a purified BHET solution; (iii) subjecting the purified BHET solution thus obtained to a second crystallization step, which yields the purified BHET in crystalline form.

The first step involves crystallizing BHET from its crude solution. Preferably, the crude BHET solution is a BHET solution in a mixture of water and MEG, coming directly from a PET glycolysis process.

The suspension of BHET in crystalline form is then filtered to obtain BHET in crystalline form and a liquid phase (mother liquor) from which the MEG can be recovered (e.g. by distillation) which can be recycled to the glycolysis process.

The BHET thus obtained in crystalline form is then redissolved, generally in water, and subjected to a purification step, to remove the contaminants deriving from the starting PET. This purification step can be carried out according to known techniques, preferably according to the process described in WO 2021/124149, already mentioned above. This purification step preferably includes an oxidation step of the crude BHET solution, followed by a treatment step with an absorbent agent (for example an activated carbon or a silica), which is capable of removing the oxidation products of the dyes or other contaminants present in the raw BHET solution.

The aqueous solution of purified BHET thus obtained can then be subjected to a second crystallization step, which yields the purified BHET in crystalline form, which appears as a solid mixed intimately with the residual solvent (water), which is called cake, in jargon.

At least one of the two crystallization steps is carried out according to the process in accordance with the present invention. Preferably, at least the first crystallization step is carried out according to the process in accordance with the present invention. More preferably both the first and second crystallization steps are carried out according to the process in accordance with the present invention.

In a preferred embodiment, the first crystallization step and/or the second crystallization step are in turn divided into two crystallization sub-steps which operate at different temperatures. In other words, the first sub-step is carried out in a first crystalliser and the second sub-step in a second crystalliser, said crystallisers being made according to the present invention. Inside the first crystallizer the temperature is higher than the temperature inside the second crystallizer. This subdivision of the crystallization process has the main advantage of smoothing the temperature drop necessary to obtain a complete crystallization of the BHET starting from the initial temperature of the inlet BHET solution, obtained from the PET glycolysis process, which is generally between 50° C. and 75° C., while at the end of crystallization the liquid phase is at a temperature around 10° C.-17° C.

1. A process for crystallizing bis(2-hydroxyethyl) terephthalate (BHET) in a continuous manner, which comprises:
introducing a BHET solution into a crystallizer which comprises a tank equipped with an impeller positioned in a lower part of the tank and coaxial to a draft tube arranged inside the tank, where the external wall of the draft tube defines, with the internal wall of the tank, an interspace within which a plurality of heat exchange plates are located;
rotating the impeller so as to generate a thrust of the solution from the bottom upwards, along the longitudinal development direction (S) of the draft tube and consequently a flow of the solution from the top downwards inside the interspace, where the solution cools on contact with the plurality of heat exchange plates in which a cooling fluid circulates, at a temperature such as to cause crystallization of the BHET with formation of a BHET crystal suspension; and
discharging the BHET crystal suspension from the crystallizer.

2. The process according to claim 1, wherein the impeller is of a reverse helix type.

3. The process according to claim 1, wherein the impeller is sized and rotated at an angular speed such as to cause a solution flow having a speed inside the interspace from 0.2 m/sec to 2.0 m/sec.

4. The process according to claim 1, wherein the heat exchange plates are positioned inside the interspace inclined according to angles (α) comprised between 10 and 50 degrees with respect to the corresponding radial direction, with reference to a transversal plane.

5. The process according to claim 1, wherein the heat exchange plates have a configuration of pillow plate heat exchangers.

6. The process according to claim 1, wherein the heat exchange plates have an outer surface treated by electropolishing.

7. The process according to claim 1, further comprising a step of cleaning the heat exchange plates, which comprises interrupting the cooling fluid flow and replacing it with a heating fluid flow.

8. The process according to claim 7, wherein the cleaning step is performed on a limited number of plates in turn.

9. The process according to claim 1, wherein the crystallizer comprises a number of heat exchange plates greater than or equal to 20.

10. The process according to claim 1, wherein inside the crystallizer the solution is at a temperature from 10° C. to 40° C.

11. The process according to claim 1, said process being subdivided into at least two crystallization steps, wherein:
a first step comprises crystallizing the BHET from a crude BHET solution, so as to obtain a suspension of BHET in a crystalline form which is then filtered to obtain the BHET in a crystalline form and a liquid phase (mother liquor); and
a second step comprises: (i) redissolving the BHET in a crystalline form obtained from the first step; (ii) subjecting the thus obtained BHET solution to a purification step, so as to obtain a purified BHET solution; and (iii) subjecting the purified BHET solution thus obtained to a second crystallization step, which yields the purified BHET in a crystalline form.

12. The process according to claim 11, wherein at least one of the two crystallization steps, is carried out in the crystallizer.

13. The process according to claim 11, wherein the first crystallization step and/or the second crystallization step are each divided into two crystallization sub-steps, wherein the first sub-step is carried out in a first crystallizer and the second sub-step in a second crystallizer, wherein the temperature inside the first crystallizer is higher than the temperature inside the second crystallizer.

14. A crystallizer for crystallizing BHET from a solution thereof, which comprises:
a tank;
a draft tube arranged inside said tank, between the internal wall of the tank and the external wall of the draft tube being defined an interspace within which a plurality of heat exchange plates are located;
an impeller positioned in a lower part of the tank, internally and coaxially to the draft tube;
a feeding duct for feeding the BHET solution into the tank; and
a discharge duct for discharging the crystallized BHET in a suspension form from the tank.

15. The crystallizer according to claim 14, wherein the impeller is of a reverse helix type.

16. The crystallizer according to claim 14, wherein each heat exchange plate has a height, along the main direction of longitudinal development, at least equal to the height of the draft tube.

17. The crystallizer according to claim 14, wherein the heat exchange plates are positioned inside the interspace inclined according to angles (α) comprised between 10 and 50 degrees with respect to the corresponding radial direction, with reference to a transversal plane.

18. The crystallizer according to claim 14, wherein the heat exchange plates have a configuration of pillow plate heat exchangers.

19. The crystallizer according to claim 14, wherein the heat exchange plates have an outer surface treated by electropolishing.

20. The crystallizer according to claim 14, wherein the heat exchange plates are present in a number greater than or equal to 20.

21. The crystallizer according to claim 14, wherein each heat exchange plate comprises an inlet conduit and an outlet conduit for a cooling fluid or for a heating fluid.

22. The crystallizer according to claim 14, which further comprises a plurality of solenoid valves, one for each heat exchange plate, configured to switch supply of each plate between a cooling fluid supply and a heating fluid supply.

23. The crystallizer according to claim 14, which further comprises a heating jacket, placed externally to the walls of the tank and able to heat the solution contained in the tank.
